(19) United States
(12) Patent Application Publication
(10) Pub. No.: US 2010/0069326 A1
(43) Pub. Date: Mar. 18, 2010

(54) Combination Therapies to Treat Cardio- and Cerebro-Vascular Disorders

(76) Inventors: Wasimul Haque, Edmonton (CA); Neil Dunwald, Edmonton (CA)

Correspondence Address: WILLIAM J BUNDREN;THE LAW OFFICE OF WILLIAM J BUNDREN, 734 LaRue Road, Millersville, MD, 21108, US

(21) Appl. No.: 12/541,886

(22) Filed: Aug. 14, 2009

Related U.S. Application Data
(63) Continuation-in-part of application No. PCT/CA2009/000175, filed on Feb. 3, 2009.
(60) Provisional application No. 61/028,542, filed on Feb. 14, 2008.

(51) Int. Cl. A61K 31/727; A61K 31/225; A61K 31/519; A61K 31/4709; A61P 9/00; A61P 35/00

(52) U.S. Cl. 514/56; 514/548; 514/262.1; 514/312

(57) ABSTRACT

The invention is compositions and methods for treating vascular disorders and/or conditions that involve administering a first agent comprising triflusal (or a triflusal analog or derivative) in combination with at least one other active agent.

This application is a continuation-in-part of International Application No. PCT/CA2009/000175, filed 13 Feb. 2009, which claims the benefit of U.S. Provisional application Ser. No. 61/028,542 filed 14 Feb. 2008. These applications are hereby incorporated by reference.

### FIELD OF INVENTION

The invention relates to methods, uses, and compositions comprising pharmaceutical compositions of at least one antiplatelet agent, 2-acetoxy-4-trifluoromethylbenzoic acid, also known as triflusal. The invention may also include other antiplatelet agents and/or agents for treating vascular disorders, including but not limited to dipyridamole or cilostazol; P2Y₁₂ inhibitors, such as clopidogrel or prasugrel; COX-2 inhibitors, such as celecoxib; and anticoagulants, such as enoxaparin. The methods, uses, and compositions of the present invention may be used for secondary prevention of stroke, and those caused by prothrombotic states induced by platelet aggregation and/or an activated coagulation cascade, among other diseases and conditions

### BACKGROUND OF INVENTION

Cardiovascular and cerebrovascular diseases are the leading cause of death in the world. Several factors that lead to cardiovascular or cerebrovascular disorders are also known to increase the susceptibility of individuals to stroke.

It is desirable to develop treatments for cerebrovascular disease, including cerebral hemorrhage, cerebral ischemia, ischaemic stroke, hemorrhagic stroke, and ischaemic reperfusion injury arising from reintroduction of blood flow following cerebral ischemia or ischaemic stroke. Cerebrovascular disease includes any abnormality of the brain resulting from a pathologic process of a blood vessel. A pathologic process of a blood vessel includes any one or more of the following: an occlusion of a blood vessel lumen by thrombus or embolus, a rupture of a blood vessel, an altered permeability of a blood-vessel wall, and increased viscosity or other change in the quality of blood.

Cerebrovascular disease is typically readily diagnosable because of how it manifests. Cerebrovascular disease typically manifests as a stroke. A stroke can be characterized as a sudden nonconvulsive, focal neurologic deficit. That is, stroke can be characterized as the death of brain tissue that results from lack of blood flow and insufficient oxygen to the brain. After heart disease and cancer, stroke is the leading cause of death in the United States.

A stroke can be ischaemic or hemorrhagic. In an ischaemic stroke, the blood supply to part of the brain is reduced or terminated either by a blood clot that blocks a blood vessel or by atherosclerosis. Reducing or terminating blood flow to the brain is known as cerebral ischemia. Cerebral ischemia can last for seconds to minutes (Transient Ischaemic attack, mini-stroke), and when cerebral ischemia occurs for more than a few minutes, infarction of brain tissue results. A blood vessel can be blocked by a blood clot that arises from thrombus or embolus. Yet cerebral ischemia can also arise from the failure of circulation and hypotension from severe and prolonged cardiac decompensation or shock.

In a hemorrhagic stroke, the brain is damaged by a blood vessel bursting, which prevents normal blood flow and allows blood to leak into an area of the brain. In some instances, the blood leaks from a small artery. When blood leaks into the brain, a hematoma is formed in the brain and blood can spread into ventricles and subarachnoid space.

In cerebral hemorrhage, blood leaks from the vessel (usually a small artery) directly into the brain forming a hematoma, and the blood spreads into the ventricles and subarachnoid space. The hematoma can cause physical disruption of the brain tissue and pressure on the surrounding brain areas. When the blood leakage stops, the hematoma can slowly disintegrate and be absorbed over a period of weeks and months.

Acute ischaemic stroke is a notoriously difficult to treat (Therapeutic Strategies For The Treatment Of Stroke, A. Richard Green and Ashfaq Shuaib, Drug Discovery Today Volume 11, Numbers 15/16 Aug. 2006). Consequently much of the clinical effort is currently directed toward strategies for preventing recurrence of stroke. Currently, there are three alternative antiplatelet medications commonly available for patients who suffer recurrent stroke: aspirin, an aspirin-extended-release dipyridamole combination (Aggrenox®, a trade mark of Boerhinger Ingelheim), and clopidogrel. Despite years of research, only one of these products (Aggrenox) is a fixed-dose combination drug. There is also strong evidence emerging that antiplatelet drug non-responsiveness is a growing problem (Am Heart J 2008; 155:591-9.).

Acetyl-4-trifluoromethyl salicylic acid (triflusal), in combination with other drugs, overcome some of these problems. Triflusal was first reported by Hauptschein in 1958 (U.S. Pat. No. 3,019,253) as an antifungal and analgesic compound. Barra, et. al., (U.S. Pat. No. 4,096,252) teaches the use of triflusal for treating abnormal platelet aggregation. The compound has been used in several trials (e.g., NEUROLOGY 2004; 62:1073-1080) for the prevention of several cardiovascular and cerebrovascular conditions, but has not gained international success as it was not thought to confer a superior benefit to aspirin, one of the most inexpensive drugs available.

As part of combination therapies, the pharmacological properties of triflusal offer potential benefits in the targeting of specific patient populations. Triflusal demonstrates a relatively benign bleeding profile as compared to aspirin and has shown evidence of increasing vasodilation. Such properties may be complementary, additive, or synergistic with those of its partner drug.

### Clinical Manifestation of Platelet/Hemostatic Cascade

When a vulnerable plaque ruptures, factors within the vessel wall, such as collagen or von Willebrand Factor are exposed to circulation and cause platelets to adhere. In response to this event, the coagulation cascade is initiated and very often the vessel becomes occluded, causing non-Q wave myocardial infarction, myocardial infarction, transient ischaemic events, and stroke. Activation of platelets has also been implicated in coronary artery disease, peripheral artery disease, unstable angina, asthma, rhinitis, chronic obstructive pulmonary disease, rheumatoid arthritis, inflammatory bowel disease, and atherosclerosis.

Aspirin is one of the most frequently used oral agents for retarding platelet aggregation for the purpose of forestalling occlusive events. It has been reported that as many as 40% of humans using standard doses of aspirin have some degree of difficulty achieving appropriate inhibition without adverse events. One key to replacing aspirin in therapy is to find agents that will, by themselves or in combination, safely and effectively provide the necessary degree of platelet inhibition.

Despite the great amount of work that has been done in investigating many new pathways to antiplatelet therapy, there are still problems. Whichever medication has been chosen, blocking platelets reduces the body's effectiveness in maintaining normal hemostasis. This tendency is often exacerbated by using drugs in the form and dosages approved for monotherapy. Also many of the products are only available for parenteral administration rendering them ineffective for preventative regimens.

Combining two antiplatelet agents with different mechanisms of action provides a substantial increase in efficacy. The combination of extended-release dipyridamole and aspirin reduced the relative risk of secondary stroke by 37% and, accordingly, use of this combination is presently recommended for secondary prevention of stroke. Importantly, the risk of major bleeding attributable to this combination therapy was no greater than that seen with aspirin alone.

The results of the Clopidogrel in Unstable Angina to Prevent Recurrent Events (CURE) trial were published (Stroke. 2001; 32:2948). This study assessed the safety and efficacy of the combination of clopidogrel and aspirin in patients with acute coronary syndromes without ST-segment elevation. Although the combination showed improved outcomes (absolute benefit of 2.1%), the attached risk (major bleeding) was also increased (absolute increased risk of 1.0%) and minor bleeding was twice that of clopidogrel alone. Many physicians assume that the combination of clopidogrel with aspirin may be substantially more effective for treating cerebrovascular disease than either agent alone. In fact, many patients are currently treated with this combination despite the absence of any substantial safety or efficacy data.

In addition to finding aspirin free combinations for treating unstable angina and stroke, the drug combinations provided here will provide alternatives useful for other vascular conditions.

### SUMMARY OF THE INVENTION

The invention relates to compositions and methods of treating a thrombotic or thromboembolic event in a patient using antiplatelet and/anticoagulant agents. In some embodiments of the invention, the compositions and methods involve aspirin-free combinations of two or more such agents. In another preferred population, the patient is aspirin resistant. In one other preferred population the patient is at risk of aspirin-induced bleeding.

Some embodiments of the invention include a composition and method of treating a thrombotic or thromboembolic event in a patient in need of such treatment comprising administering a therapeutically effective amount of triflusal and administering a therapeutically effective amount of at least one additional agent.

Some embodiments of the invention may include treating post-thrombotic or post-thromboembolic event.

A triflusal/dipyridamole or triflusal/cilostazol are drug combinations in which both components act upon the same pathways in an additive or synergistic manner. Triflusal is known to have a lower haemorrhagic effect than aspirin as well as to act as a phosphodiesterase inhibitors. The latter effect is additive or synergistic with dipyridamole or cilostazol. Cilostazol is not known to cause headaches like dipyridamole. Both combinations are aspirin free so that aspirin non-responders may benefit. Patients who have suffered intermittent claudication or ischaemic stroke will benefit from reduced frequency of bleeding events and the vasodilatory synergies.

In particular, the invention involves the use of combinations of antiplatelet agents to improve over current therapies. In another object of the invention, the inventors seek to prevent cardiovascular and cerebrovascular diseases from occurring and ameliorating the consequences of pathological cardiovascular and cerebrovascular events.

Several studies have suggested that up to 45% of patients undergoing aspirin-mediated antiplatelet therapy may not respond to aspirin with sufficient inhibition of platelet function. Accordingly, another object is to create aspirin-free alternatives for these groups of patients. A specific object of this invention is to provide alternate medications that will improve neuroprotective and vasodilating properties as compared to products on the market. A final objective of this invention is to provide alternate medications that reduce bleeding events.

Triflusal/dipyridamole for secondary prevention of stroke For the prevention of recurrent stroke, triflusal/dipyridamole may have a theoretically superior neuroprotection profile, a potentially better inhibition of neurological cyclooxygenase, COX-3, and lower vasoconstrictive properties than either aspirin or aspirin in combination with dipyridamole. Triflusal is five times more potent than aspirin as inhibitor of cAMP phosphodiesterase. The synergic or additive effects on cAMP levels may allow the combination to contain a reduced dose of dipyridamole than that used in the current product on the market. Triflusal also has favorable neuroprotective effects in stroke patients by eliciting greater inhibitory effects of cytokine IL-6.

Triflusal (2-acetoxy-4-trifluoromethylbenzoic acid), a 4 fluoromethyl derivative of salicylate acid, and it's active metabolite 2-hydroxy-4-trifluoromethylbenzoic acid (HTB) are direct inhibitors of cyclooxygenase-2 (COX-2) and indirect inhibitors of NF-κ B (Bayon et al, 1999), whereas aspirin is a COX-I/COX-2 inhibitor. Both triflusal and aspirin have properties that may help protect the brain against neuroinflammatory and apoptotic mechanisms in cerebral infarction. It has been demonstrated in the rat middle cerebral artery occlusion (MCAO) model of stroke that a dose of 30 mg/kg for triflusal and aspirin were equally effective in reducing infarct size (Whiteheads, et. al, 2007).

Dipyridamole has antiplatelet and vasodilator properties with a mechanism of action that may be related to inhibition of platelet phosphodiesterase, stimulation of prostacyclin, or inhibition of adenosine uptake (Sudlow, et al, 2005). It has been demonstrated that the combination of aspirin and dipyridamole is more effective than aspirin alone in the secondary prevention of stroke (Halkes et al, 2008). Thus, it is possible that a combination of dipyridamole and triflusal might be an effective treatment for prevention of recurrent stroke.

Triflusal/clopidogrel combination to treat unstable angina or non-ST elevation myocardial infarction.

Patients require more powerful platelet inhibition in these circumstances. While one skilled in the art may arrive at the notion of combining aspirin and clopidogrel therapies to accomplish this inhibition, trials of aspirin/clopidogrel have shown efficacy but also increased the risk of bleeding to the extent that the risks of treatment outweigh the benefits in most cases. Triflusal's relatively benign profile with respect to hemorrhage makes combining it with a P2Y₁₂ inhibitor such as clopidogrel an attractive alternative to clopidogrel alone or in combination with aspirin in the acute setting.

Triflusal/Low-Molecular-Weight Heparin Combination for the Treatment of Patients with Atrial Fibrillation:

While this combination may also find use in treating acute coronary syndrome, deep-vein thrombosis, pulmonary embolism, and cardiopulmonary bypass surgery, patients with chronic atrial fibrillation have few good options for long-term preventive therapy. Those with a low risk are often treated with anticoagulant plus aspirin while higher risk patients are treated with warfarin and an anticoagulant. Patients in the low risk group may benefit from Triflusal/clopidogrel combination administration while those in an acute setting group should benefit from Triflusal/low-molecular-weight heparin combination.

Triflusal/Celecoxib for Treatment of Patients with Peripheral Artery Disease:

Vioxx (valdecoxib), Bextra (Rofecoxib), Arcoxia (etoricoxib) and Celebrex (celecoxib) are four selective COX-2 inhibitors to make it to market (Comp. Ther. 2006; (32(4) incorporated by reference in its entirety). The former two have been withdrawn from market because of reports of adverse cardiovascular events. One theory, to which the inventors do not wish to be bound, has it that endothelial cells lining blood vessels express COX-2, and, by selectively inhibiting it, prostaglandins (specifically PGI₂; prostacyclin) are downregulated relative to thromboxane levels. Since COX-1 in platelets is unaffected by the monotherapy, the protective anti-coagulative effect of PGI₂ is decreased, increasing the risk of thrombus and other circulatory problems. This combination will have the antiplatelet activity of triflusal, the anti-inflammatory properties of COX-2 inhibition, as well as the analgesic properties of both. The combination of triflusal and celecoxib should provide inflammatory relief at significantly lower doses of each entity on its own; consequently should provide a lower risk therapy for chronic conditions such as PAD, neuropathies associated with vasculitis or inflammation of the blood vessels, or arthritic conditions

### DETAILED DESCRIPTION OF THE INVENTION

The invention is compositions and methods for treating vascular disorders and/or conditions that involve administering a first agent comprising triflusal (or a triflusal analog or derivative) in combination with at least one other active agent (e.g., a second active agent). In some embodiments of the invention, the second active agent may be an anti-platelet agent. As used herein, an antiplatelet agent affects platelet function, typically by inactivating platelets, or inhibits or reducing platelet activation, or by reducing or inhibiting platelet adhesion and/or aggregation. The preferred triflusal derivative is HTB.

In combination, as used herein, refers to administering the agents concurrently, sequentially, or mixed together. It is intended that regardless of the relative timing of administering both agents, each agent works together to provide a therapeutic benefit for the patient, such as treating a condition or disease, or relieving one or more symptoms, or treating one or more side-effects of the other agent.

Specifically, the inventors have created four combination drug products, a triflusal/clopidogrel combination to treat unstable angina or non-ST elevation myocardial infarction, a triflusal/low-molecular-weight heparin combination for the treatment of patients with atrial fibrillation, a triflusal/celecoxib for treatment of patients with peripheral artery disease, and a triflusal/phosphodiesterase inhibitor combination for the secondary prevention of stroke.

In accordance with the present invention, the second active agent may be any additional agent that provides a therapeutic benefit. Such additional agents include but are not limited to HTB; a GPIIb/IIIa inhibitor such as aciximab, eptifibatide, tirofoban, and lamifiban; an ADP receptor antagonist, such as ticlopidine, prasugrel, or clopidogrel; or a phosphodiesterase inhibitor, such as dipyridamole or cilostazol, and Aggrenox®; or a selective serotonin reuptake inhibitor.

Some embodiments of the invention involve compositions and methods of treating a thrombotic or thromboembolic event in a patient in need of such treatment comprising administering a therapeutically effective amount of a first agent (e.g., triflusal or HTB) in combination with a therapeutically effective amount of an anticoagulant.

Some embodiments of the invention include administering a first active agent (e.g., triflusal or HTB), an anticoagulant, and a thrombolytic agent.

One skilled in the art will recognize that a wide range of anticoagulant compounds may be used. Exemplary anticoagulant compounds include, but are not limited to unfractionated heparin, hirudin, an antithrombin (e.g. human antithrombin III), sulodexide, or a low molecular weight heparin (e.g., bemiparin, dalteparin, enoxaparin, nadroparin, pamaparin, reviparin, tinzaparin).

Some embodiments of the invention include administering a first agent in combination with a low molecular weight heparin. In this embodiment enoxaparin is an exemplary low-molecular-weight heparin. In some of these embodiments of the invention, triflusal and a low-molecular-weight heparin such as enoxaparin may be combined for the treatment of acute coronary syndrome and/or to treat atrial fibrillation.

Some embodiments of the invention include administering the first active agent (e.g., triflusal or HTB) in combination with a P2Y₁₂ inhibitor. The P2Y₁₂ inhibitor may be selected from any of the exemplary compounds as follows: clopidogrel, ticlopidine, or prasugrel. The preferred P2Y₁₂ inhibitor is clopidogrel. In some embodiments of the invention, the first agent and the P₂Y₁₂ inhibitor such as clopidogrel are combined for the treatment of a coronary condition, such as acute coronary syndrome, unstable angina, and/or non-ST elevation myocardial infarction.

Some embodiments of the invention include administering the first active agent (e.g., triflusal or HTB) in combination with a selective COX-2 inhibitor, e.g., one that does not inhibit COX-1. In some embodiments, the COX-2 inhibitors are not selective inhibitors. Exemplary non-selective inhibitors include, but are not limited to aspirin, ibuprofen, naproxen, indomethacin, and diclofenac.

In some embodiments of the invention the selective COX-2 inhibitors may be selected from any of the exemplary compounds as follows: valdecoxib, rofecoxib, eetoricoxib and celecoxib. The preferred COX-2 inhibitor is celecoxib. In some embodiments of the invention, triflusal or HTB and celecoxib are combined for the treatment of peripheral artery disease.

In some embodiments of the invention, the first agent combined with the second agent may be used to inhibit thrombosis in patients. In a preferred aspect of this invention the first active agent is triflusal or HTB. In these embodiments of the invention, the second active agent may be a phosphodiesterase inhibitor, including but not limited to dipyridamole or cilostazol. The dipyridamole may be formulated to achieve extended release. In these embodiments, the combination of triflusal and dipyridamole may be used to prevent stroke and/or to treat stroke patients at risk of aspirin-induced bleeding. In some embodiments of the invention, triflusal and dipyridamole may be used in patients who are non-responders to aspirin, or aspirin resistant; or in the secondary prevention of stroke in patients who are at risk of aspirin-induced bleeding, are non-responders, or are aspirin resistant;

In other embodiments, the first agent combined with the second agent (e.g., triflusal/dipyridamole) may be used for diabetic hypertensive patients, in type II diabetics (non-insulin dependent diabetes mellitus, NIDDM, or adult-onset diabetes).

One skilled in the art will recognize that various doses and frequency of doses may be used. In some embodiments of the invention, oral combinations may be administered in fixed dose formulations. In some embodiments of the invention, one or more of the agents in the combination will have a lower dose than the dose used in monotherapy. The dose used to achieve a beneficial result is any dose and frequency of administration sufficient to provide a therapeutic benefit for the patient. One skilled in the art will recognize that many factors may be involved in determining a proper dosage, including but not limited to the size and weight of the patient, the particular active agent or combination of active agents being used, and the age of the patient.

The recommended antithrombotic dose of triflusal is 10 mg/kg/day. At this dose, triflusal does not confer significant neuroprotection in the MCAO model (12.56% reduction) but 30 mg/kg/day does (43.95% reduction) (Stroke. 2007; 38:381-387.). While not intending to be limited to a particular dose or dose range, the inventors have found that when used in combination with dipyridamole a dose about 10 mg/kg/day of triflusal significantly reduces infarct size (37.03% reduction) as compared to a 30/mg/kg/day dose (23.62%). The inventors extrapolate that at doses significantly less than 10 mg/kg day of triflusal, the combination will continue to exhibit neuroprotection. Accordingly, triflusal combinations may incorporate in a wide range of doses to achieve therapeutic benefit. In particular, neuroprotective triflusal combinations for may employ a low dose, That does may be effective in the range from 1 mg/kg/day to 15 mg/kg/day of triflusal and most likely will be effective in the range of 2.5 mg/kg/day to 7.5 mg/kg/day. One skilled in the art will recognize that when such synergies can be achieved, patients not only benefit from the novel combination, they also benefit from requiring less active ingredient to achieve an equivalent effect. The lower doses also reduce the potential for unintended effects such as bleeding.

The composition of the invention may include a pharmaceutically acceptable carrier, adjuvant or vehicle that may be administered to a subject, together with a combination of the present invention, and which does not destroy the pharmacological activity of the combination. Pharmaceutically acceptable carriers, adjuvants and vehicles that may be used in the compositions of the present invention include, but are not limited to, the following: ion exchangers, alumina, aluminum stearate, lecithin, self-emulsifying drug delivery systems (“SEDDS”) such as d(-tocopherol polyethyleneglycol 1000 succinate), surfactants used in pharmaceutical dosage forms such as Tweens or other similar polymeric delivery matrices, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Exemplary compositions include those formulating the combination(s) of the invention with fast dissolving diluents such as mannitol, lactose, sucrose and/or cyclodextrins. Also included in such formulations may be high molecular weight excipients such as celluloses or polyethylene glycols. Such formulations may also include an excipient to aid mucosal adhesion such as hydroxy propyl cellulose, hydroxy propyl methyl cellulose, sodium carboxy methyl cellulose, maleic anhydride copolymer, and agents to control release such as polyacrylic copolymer. Lubricants, glidants, flavors, coloring agents, and stabilizers may also be added for ease of fabrication and use. Cyclodextrins such as .alpha.-, .beta.- and .gamma.-cyclodextrin, or chemically modified derivatives such as hydroxyalkylcyclodextrins, including 2- and 3-hydroxypropyl-.beta.-cyclodextrins, or other solubilized derivatives may also be used to enhance delivery of the combinations of the present invention.

The combination may be administered by any suitable means, for example, orally, such as in the form of tablets, capsules, granules or powders; sublingually; buccally; parenterally, such as by subcutaneous, intravenous, intramuscular, or intrasternal injection or infusion techniques; nasally such as by inhalation spray; topically, such as in the form of a cream or ointment; or rectally such as in the form of suppositories; in dosage unit formulations containing non-toxic, pharmaceutically acceptable vehicles or diluents. The present combinations may, for example, be administered in a form suitable for immediate release, extended release or combinations thereof. Immediate release or extended release may be achieved by the use of suitable pharmaceutical compositions comprising the present combinations, or, particularly in the case of extended release, by the use of devices such as subcutaneous implants or osmotic pumps. The combinations may be delivered individually or in dually as prodrugs. The present combinations may also be administered liposomally. For example, the active substance can be utilized in a composition such as tablet, capsule, solution or suspension or in topical form. They may be combined in a conventional manner with a physiologically acceptable vehicle or carrier, excipient, binder, preservative, stabilizer, flavor, etc., or with a topical carrier.

### DEFINITIONS

“Anticoagulant” refers to a molecule that interferes with the non-cellular participants of a coagulation cascade. Accordingly, they have very little direct effect on platelets. Exemplary anticoagulants include Vitamin K antagonists (e.g. warfarin (coumadin), acenocoumarol or phenprocoumon); Heparin, heparin combinations, and derivative substances (e.g. unfractionated heparin, heparin and hirudin, danaparoid); low molecular weight heparin (e.g., bemiparin, dalteparin, enoxaparin, nadroparin, parnaparin, reviparin, tinzaparin sulodexide); Synthetic pentasaccharide inhibitors of factor Xa; and antithrombin (e.g. human antithrombin III),

“Antiplatelet agent” refers to a pharmaceuticals used in primary and secondary prevention of thrombotic events that decreases platelet aggregation and inhibit thrombus formation. The importance of inhibiting platelets lies in the fact that platelet adhesion to the endothelium is the initiating event in primary hemostasis. Accordingly, antiplatelet agents are effective in the circulation where anticoagulants have little effect. Drugs considered to be antiplatelet agents are cyclooxygenase inhibitors (e.g. aspirin, triflusal); adenosine diphosphate (ADP) receptor inhibitors—(e.g. clopidogrel, Ticlopidine); phosphodiesterase inhibitors (e.g. cilostazol); glycoprotein IIB/IIIA inhibitors (e.g., abciximab, eptifibatide, tirofiban, defibrotide); and adenosine reuptake inhibitors (e.g. dipyridamole). The reader should note that antiplatelet agents have more than one therapeutic property. For example, it is possible for one agent to be both an antiplatelet agent and a vasodilator.

“Aspirin” refers to a white, crystalline acetylated derivative of salicylic acid, CH₃COOC₆H₄COOH, derived from salicylic acid and commonly used in tablet form to relieve pain (especially from headache, rheumatism, gout, neuralgia, etc.), fever, and inflammation. It is also used as an antiplatelet agent to slow clotting of the blood by inhibiting the aggregation of platelets. In long-term antiplatelet therapy patients are normally prescribed enteric-coated aspirin to forestall gastrointestinal bleeding.

“Aspirin Resistance” refers to the phenomenon whereby aspirin does not have as strong an effect on platelets as for others. Women are reportedly more likely to be resistant than men but there is currently no accepted method of determining who is resistant. The phenomenon is also known as “aspirin insensitivity”.

“Aspirin Non-Responders” refers to those patients who do not exhibit significant antiplatelet activity when treated with the standard dose.

“Atherosclerosis” is a chronic inflammatory disease affecting arterial blood vessels. It is a response in large part due to the deposition of lipoprotein plaques (comprised of plasma proteins, cholesterol, triglycerides, calcium and scar tissue) on the walls of arteries.

“Coronary Artery Disease (CAD)” is atherosclerosis of one or more of the coronary arteries.

“COX-2 inhibitor” refers to a molecule that inhibits the COX-2 enzyme to a higher degree than it inhibits the COX-1 enzyme. Ibuprofen, naproxen, indomethacin, diclofenac are drugs that perform this function. A selective COX-2 inhibitor inhibits the COX-2 enzyme while have little or no effect on the COX-1 enzyme. Examples of selective COX 2 inhibitors are valdecoxib, rofecoxib, eetoricoxib and celecoxib.

“Heart attack (myocardial infarction, AMI, MI)” is a medical condition that occurs when the blood supply to a part of the heart is interrupted, most commonly due to rupture of a vulnerable plaque in a coronary artery. A heart attack may also occur when a coronary artery temporarily contracts or goes into a severe spasm, effectively shutting off the flow of blood to the heart. In either case, the resulting ischemia or oxygen shortage causes damage and potential death of heart tissue.

“Intermittent claudication” refers to muscle pain (ache, cramp, numbness or sense of fatigue) which occurs during exercise and is relieved by a short period of rest.

“Neuroprotective” and similar words refer to interventions protecting brain tissue from damage, in particular, protecting neurons from cell death or degeneration. Within the context of ischemic stroke or TIA, the intervention must slow the cascade of molecular events that occur in oxygen deprived brain tissue.

For neuroprotection to occur, it may be necessary for to simultaneously target multiple aspects of this cascade and combination drugs are particularly attractive for this purpose. While functional outcomes from intervention are the ultimate test of neuroprotection, for the purposes of definition herein we use infarct size in animal models to gauge the extent of neuroprotective effects.

“P2Y₁₂ inhibitor” refers to a molecule that blocks the P2Y₁₂ protein from acting on a chemoreceptor for adenosine diphosphate (ADP). It is found on the surface of blood platelet cells. In this application, P2Y₁₂ inhibitors are exemplified by clopidogrel, ticlopidine, or prasugrel.

“Patients at risk of aspirin-induced bleeding” refers to patients that have at least one factor that would place them at risk of bleeding due to aspirin-mediated antiplatelet therapy. These risk factors are generally: prior gastrointestinal bleeding events (e.g. ulcers); increased age; use of anticoagulants such as warfarin; use of corticosteroids; and concomitant use of increased dose or multiple non-steroidal anti-inflammatory drugs.

“Peripheral Artery Disease (PAD)” is a condition similar to CAD but occurring in arteries outside of the heart or brain. In PAD, fatty deposits build up along the inner linings of the artery walls. These blockages restrict blood circulation, mainly in arteries leading to the kidneys, stomach, arms, legs, and feet. People with PAD often exhibit plaques in the arteries of the heart and brain. Because of this association, most people with PAD have a higher risk of death from heart attack and stroke.

“Strokes” are classified as either ischaemic or hemorrhagic, the former accounting for approximately 80% of all events. “Ischaemic stroke”, similar to heart attack, is caused by an interruption of blood flow to the brain. The resulting lack of oxygen supply causes damage to brain tissue. “Ischaemic strokes” have two causes. “Thrombotic strokes” are caused by a blood clot that forms in an artery directly leading to the brain. Embolic strokes occur when a clot develops somewhere else in the body and travels through the blood stream to the brain. “Hemorrhagic stroke”—About 20% of strokes in Americans are hemorrhagic which means they are characterized by uncontrolled bleeding in the brain. This “flooding” of the brain kills brain cells. Subarachnoid hemorrhage is uncontrolled bleeding on the surface of the brain, in the area between the brain and the cranium. Intracerebral hemorrhage occurs when an artery deep within the brain ruptures. Both types of hemorrhage can be caused by structural problems with the blood vessels in the brain.

A thrombotic or thromboembolic event includes but is not limited to the following: atrial fibrillation, acute coronary syndrome including, unstable angina, acute myocardial infarction, ischaemic stroke, acute coronary ischaemic syndrome, thrombosis, thromboembolism, peripheral artery disease, deep vein thrombosis, arterial thrombosis of any vessel, catheter thrombotic occlusion, thrombotic occlusion and reocclusion, transient ischaemic attack, first or subsequent thrombotic stroke.

“TIA”, or “Transient Ischaemic Attack”—also known as a “mini-stroke”—is caused by a temporary interruption of blood flow to the brain. The symptoms (warning signs) are similar to an ischaemic stroke except they go away within a few minutes or hours. A TIA is an important indicator of full-blown stroke risk; however, people frequently have a TIA without even knowing it. This detection problem complicates estimating the true size of the affected population.

“Vascular condition” refers to a pathology of or about the vasculature of the circulatory system. Specific exemplary conditions include Coronary Artery Disease (CAD); Acute Coronary Syndrome (ACS), including unstable angina and non-ST-elevated myocardial infarction; Acute Coronary Ischaemic Syndrome; first or subsequent thrombotic Stroke; Transient Ischaemic Attack (TIA); Peripheral Artery Disease (PAD); Deep Vein thrombosis (DVT); Atherosclerosis; Atrial Fibrillation; catheter thrombotic occlusion; thrombotic occlusion and reocclusion; and arterial thrombosis of any vessel.

“Vasodilator” refers to a drug or chemical that relaxes the smooth muscle in blood vessels, which causes them to dilate. This group includes Alpha-adrenoceptor antagonists (alpha-blockers); Angiotensin converting enzyme (ACE) inhibitors; Angiotensin receptor blockers (ARBs); β₂-adrenoceptor agonists; Calcium-channel blockers (GCBs); Centrally acting sympatholytics; Direct acting vasodilators; Endothelin receptor antagonists; Ganglionic blockers; Nitrodilators; Phosphodiesterase inhibitors; and Potassium-channel openers. Most vasodilators exhibit pharmacological properties as mentioned above but dipyridamole or cilostazol also act as antiplatelet agents.

Triflusal is 2-acetoxy-4-trifluoromethylbenzoic acid, generally having the following formula:

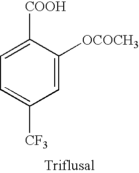

HTB is hydroxyl trifluoromethyl benzoic acid, a metabolite of Triflusal, and generally having the following formula:

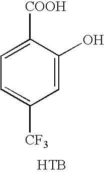

Dipyridamole is 2-{[9-(bis(2-hydroxyethyl)amino)-2,7-bis(1-piperidyl) 3,5,8,10 tetrazabicyclo[4.4.0]deca-2,4,7,9,11-pentaen-4-yl]-(2-hydroxyethyl)amino}ethanol

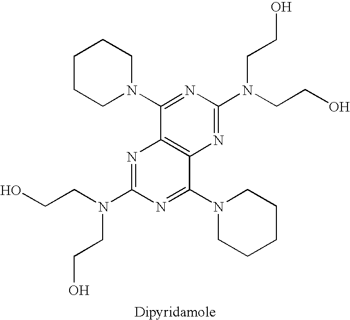

Cilostazolis 6-[4-(1-cyclohexyl-1H-tetrazol-5-yl)butoxy]-3,4-dihydro-2(1H)-quinolinone

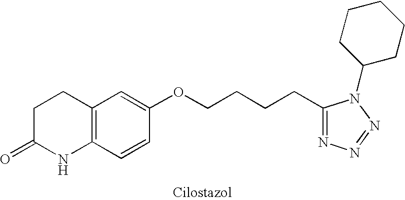

Clopidogrel is (+)-(S)-methyl 2-(2-chlorophenyl)-2-(6,7-dihydrothieno[3,2-c]pyridin-5(4H)-yl)acetate which are typically used to inhibit platelet aggregation.

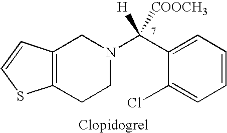

Celecoxib and other NSAIDS described in publications such as U.S. Pat. No. 6,417,204, include 4-[5-(4-methylphenyl)-3-(trifluoromethyl)pyrazol-1-yl]benzenesulfonamide which are typically used to control pain and inflammation.

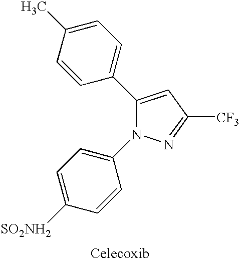

### REFERENCES

De Cristobal J, Moro M A, Davalos A, Castillo J, Leza J C, Camarero J, Colado M I, Lorenzo P, Lizasoain I. Neuroprotective effect of aspirin by inhibition of glutamate release after permanent focal cerebral ischaemia in rats. J Neurochem 2001 October; 79(2):456-9.
Khayyam N, Thavendiranathan P, Carmichael F J, Kus B, Jay V, Burnham W M. Neuroprotective effects of acetylsalicylic acid in an animal model of focal brain ischemia. Neuroreport 1999 Feb. 5; 10(2):371-4.
Riepe M W, Kasischke K, Raupach A. Acetylsalicylic acid increases tolerance against hypoxic and chemical hypoxia. Stroke 1997 October; 28(10):2006-11.
Grilli M, Pizzi M, Memo M, Spano P. Neuroprotection by aspirin and sodium salicylate through blockade of NF-kappaB activation. Science 1996 Nov. 22; 274(5291): 1383-5.
Bayon Y, Alonso A, Sanchez C M. 4-trifluoromethyl derivatives of salicylate, triflusal and its main metabolite 2-hydroxy-4-trifluoromethylbenzoic acid, are potent inhibitors of nuclear factor kappaB activation. Br J Pharmacol 1999 March; 126(6): 1359-66.
Sudlow, C What is the tole of dipyridamole in long-term secondary prevention after an ischaemic stroke or transient ischemia? Can. Med. Assoc. J. 2005 October; 173(9):1024-1026.
Halkes P H A, Gray L J, Bath P M W, Diener H-C, Guiraud-Chaumeil B, Yatsu F M, Algra A. Dipyridamole plus aspirin versus aspirin alone in the secondary prevention after TIA or stroke: a meta-analysis by risk. J Neurol Neurosurg Psychiatry 2008 June; (Epub ahead of print).
Whiteheads. N, Bayona N A., Cheng G, Allen G V., Hachinski V. C, and Cechetto D. F. (2007) Effects of triflusal and aspirin in a rat model of cerebral ischemia. Stroke. 38, 381-387.

This invention will be further characterized by the following examples. These examples are not meant to limit the scope of the invention, which has been fully set forth in the foregoing description. Variations within the scope of the invention will be apparent to those skilled in the art.

### EXAMPLES

### Example 1

### Low Dose Triflusal/Dipyridamole Combination for Secondary Prevention of Stroke

### Rat Stroke Model

Male Wistar rats (265-360 g) were randomly divided into four treatment groups (n=5-9 for each group). Prior to and following surgery rats were housed in single cages (12/12 h light/dark cycle) and were fed at libitum. Prior to surgery, all rats were anaesthetized with a single dose of sodium pentobarbital (60 mg/kg ip) and placed in a David Kopf stereotaxic apparatus. Rectal temperatures were monitored and maintained constant at 37° C. by a heating pad while the rats were under anesthesia. The right MCA was exposed. The MCA was permanently occluded at 2 points using thermal coagulator, one above and one below the inferior cerebral vein. Removal of a portion of the bone and exposure and ligation of the MCA was done with the aid of an operating stereomicroscope. Immediately following surgery, rats received one of the following treatments: 30 mg/kg triflusal or 30 mg/kg triflusal and 200 mg/kg dipyridamole or 10 mg/kg triflusal and 200 mg/kg dipyridamole or vehicle only for next three days.

### Tissue Processing

Three days after surgery, all animals were euthanized via pentobarbital overdose and perfused transaortically first with saline which was immediately followed by a low perfusion for about 1 h with 2% solution of triphenyl-tetrazolium chloride (TTC) that is followed by 4% formaldehyde (pH 7.4). The brains were removed and coronal sections (1 mm) were cut for infarct measurement. Investigator was blind to the sample treatment.

### Infarct Volume Assessment

Serial brain sections were examined and the areas of infarcted tissue were measured (SigmaScan Pro 5.0, SPSS Inc., Chicago Ill., USA). In addition, the hemispheric areas of each tissue section were measured to account for any brain swelling that might have occurred following cerebral ischemia. The volume of the infarct was calculated in mm³ by integrating the infarct sizes for each of the tissue sections that contained infarcted tissue. The 1 mm sections were photographed using a Nikon digital camera.

### Data Analysis

Results are expressed as mean±SEM of each measure. The volumes of the infarcts for each of the groups were compared by Student's unpaired two-tailed t tests. Significance was set at p≦0.05. Correlations between parameters were tested by linear regression analysis

It appears that triflusal either alone or in combination with dipyridamole when administered concurrent with cerebral ischemia decreased the size of the infarct compared with vehicle treated animals when assessed 3 days after MCAO. The decrease in infarct volume was up to 30.72% (by triflusal alone at 30 mg/kg), 23.62% (by 30 mg/kg triflusal and 200 mg/kg dipyridamole) and 37.03% (by 10 mg/kg triflusal and 200 mg/kg dipyridamole), respectively. The 23-37% decrease in infarct volume after only three-day treatment is indicative of the neuroprotective effects of triflusal alone as well as in combination with dipyridamole in stroke.

Briefly, the unilateral occlusion of the right MCAO resulted in circumscribed infarcts restricted to the right cortex and striatum. No injury was observed in the contralateral hemisphere. The infarct volume in rats receiving vehicle treatment immediately after MCAO was 106.22±10 mm³, in the animals treated with triflusal alone (30 mg/kg) was 73.59±7.0 mm³, in the animals treated with 30 mg/kg of triflusal combined with 200 mg/kg dipyridamole was 81.13±5.3 mm³ and in the animals treated with 10 mg/kg of triflusal combined with 200 mg/kg dipyridamole was 62.58±4.0 mm³ (Table 1).

What these results show is that the combination of triflusal with dipyridamole is just as effective at neuroprotection as triflusal or aspirin (Stroke. 2007; 38:381-387). Although we cannot yet say is that it is more protective, we know from many hundreds of other investigations in the literature that a 30% reduction in infarct size at 3 days may be the most that can be expected.

The effect of triflusal on infarct reduction was not significantly different than the combined effects of lower and higher doses of triflusal with dipyridamole. This suggests that both regimes of treatment are equally effective and that in fact the numbers of animals (N) in each group may account for the non-significant difference in infarct size. A larger group size might nullify this apparent difference. Moreover, triflusal at 30 mg/kg either alone or in combination with dipyridamole produced what is likely the maximum neuroprotection in this type of stroke model, however, the combination of triflusal with dipyridamole shows synergism when lower doses of triflusal were used.

The preliminary results suggest that triflusal and dipyridamole is at least as effective in neuroprotection as aspirin and dipyridamole. Taken together with literature results where it indicates that triflusal has a lower haemorrhagic effect than aspirin and is a more direct inhibitor of NF□B, the inventors speculate that triflusal is a better drug than aspirin to combine with dipyridamole for the prevention of stroke.

As previously demonstrated, triflusal is neuroprotective in our model of stroke resulting in a 30% decrease in infarct size/volume (the terms infarct size and infarct volume are used interchangeably). This is an excellent decrease in infarct size representing considerable neuroprotection.

Our results, however, allow us to state that a combination of triflusal and dipyridamole at lower concentrations is an effective treatment strategy based on their mechanisms of neuroprotection.

TABLE 1
Infarct Volume measurement 3 days after unilateral MCAO

| | Infarct volume | Infarct reduction |
|---|---|---|
| Treatment | (mm³) | (%) |
| Control (Vehicle) | 106.22 ± 10 | 0 |
| Triflusal (30 mg/kg) | 73.59 ± 7.0 | 30.72 ± 6.6 |
| Triflusal (30 mg/kg) and | 81.13 ± 5.3 | 23.62 ± 4.95 |
| Dipyridamole (200 mg/kg) | | |
| Triflusal (10 mg/kg) and | 62.58 ± 4.0 | 37.03 ± 7.19 |
| Dipyridamole (200 mg/kg) | | |

Although embodiments of the invention have been described above, it is not limited thereto, and it will be apparent to persons skilled in the art that numerous modifications and variations form part of the present invention insofar as they do not depart from the spirit, nature and scope of the claimed and described invention.

1. A pharmaceutical composition for treating one or more vascular disorders comprising triflusal or one of its metabolites in combination with at least one second active agent selected from the group consisting of: a low molecular weight heparin, a GPIIb/IIIa inhibitor, an ADP receptor antagonist, and a phosphodiesterase inhibitor.

2. The composition of claim 1 wherein the combination is neuroprotective.

3. The composition of claim 2 wherein the neuroprotection is synergistic

4. The composition of claim 1 wherein the neuroprotective dose of triflusal in the MCAO model is between about 1 mg/kg/day and about 15 mg/kg/day.

5. The composition of claim 2 wherein the phosphodiesterase inhibitor is selected from the group consisting of dipyridamole and cilostazol.

6. The composition of claim 1 for treatment of arterial thrombosis.

7. The composition of claim 6 for the prevention of arterial thrombosis.

8. The composition of claim 7 wherein the vascular condition is TIA, stroke or risk of stroke.

9. A method of treating one or more vascular disorders or conditions comprising administering a first agent selected from the group consisting of triflusal and one of its metabolites, and a second agent active agent selected from the group consisting of: a low molecular weight heparin, a GPIIb/IIIa inhibitor, an ADP receptor antagonist, and a phosphodiesterase inhibitor.

10. The method of claim 9 wherein the phosphodiesterase inhibitor is selected from a group consisting of dipyridamole and cilostazol.

11. A method of claim 9 wherein the agents are administered concurrently.

12. A method of claim 9 wherein treating comprises providing neuroprotection.
